(19) United States
(12) Patent Application Publication
(10) Pub. No.: US 2026/0346242 A1
(43) Pub. Date: Nov. 5, 2026

(54) LATERAL FLOW TEST SYSTEMS AND METHODS

(71) Applicant: ÉCLATERAL LIMITED, Chepstow (GB)

(72) Inventors: Benjamin James EDWARDS, Chepstow (GB); Uros ZUPANCIC, Chepstow (GB)

(21) Appl. No.: 19/487,981

(22) PCT Filed: May 23, 2024
(86) PCT No.: PCT/GB2024/051336

§ 371 Date: Nov. 25, 2025

(30) Foreign Application Priority Data
May 31, 2023 (GB) 2308137.5

(51) Int. Cl. B01L 3/00

(52) U.S. Cl. CPC B01L 3/5023; B01L 2200/16; B01L 2300/0627; B01L 2300/0645; B01L 2300/069; B01L 2300/0825

(57) ABSTRACT

The present disclosure relates to a device for performing a lateral flow test on a liquid sample, comprising: a test strip which comprises: a membrane having a first capturing reagent disposed on a first surface at a test position, the first capturing reagent being configured to capture an analyte in the liquid sample; a sample pad disposed on the first surface at a first end of the membrane spaced apart from the test position, the sample pad being configured to receive the liquid sample; and a conjugate pad disposed on the first surface between the sample pad and the test position, the conjugate pad having deposited thereon a catalytic labelling reagent, the catalytic labelling reagent being configured to chemically bind to the analyte; an activation layer disposed over the membrane arranged to contact the first surface upon activation, the activation layer having deposited thereon at least a component of a catalyst substrate, the catalyst substrate being capable of chemically reacting with the catalytic labelling reagent upon activation; and a support structure configured, when the device is in an inactive state, to affix the test strip and the activation layer such that the activation layer is separated from the membrane.

### FIELD

The present disclosure relates generally to lateral flow test devices and methods.

### BACKGROUND

Immunosensors based on the antibody-antigen immunocomplex formation are typically reliant on the catalytic function of labelling molecules, e.g. an enzymes, conjugated to an antibody. For example, an assay like the enzyme-linked immunosorbent assay (ELISA) make use of an enzymatic reaction to catalyse the conversion of a substrate into a product measurable by absorbance as a consequence of an analyte being present in a liquid sample. Herein, substrate refers to a reactant that is consumed during a catalytic or enzymatic reaction. In an assay such as ELISA, the enzyme can amplify the measurable signal while retaining specificity. However, the assay requires multiple steps (e.g. sample taking, first wash, reaction with labelling molecule, second wash, substrate introduction, stop solution introduction) to be performed in order to complete the assay, as the enzyme substrate can only be introduced to the sample-labelling molecules mixture after they have reacted.

One type of widely and commonly used immunosensors is a single-step lateral flow test (LFTs), in which the test is completed in a single step by a user depositing a sample, or a sample-buffer mixture, onto the LFT. A lateral flow test strip generally comprises a plastic backing card, which supports a membrane (e.g. a porous hydrophilic membrane), where an analyte-specific capture antibody or a set of analyte-specific capture antibodies are immobilised along a test line. Upstream of the membrane is a conjugate pad, which is typically impregnated with a second set of analyte-specific detection antibodies, which are conjugated for example with a latex bead or a gold nanoparticle (AuNP) label. Provided upstream of the conjugate pad is a sample pad, which receives a liquid sample applied by a user. The strip typically ends with an absorption pad that encourages the flow of the liquid sample through capillary action, and acts as a waste reservoir while preventing backflow. When a user deposits a sample on the sample pad, the sample travels towards the conjugate pad and dissolves the antibody-AuNP conjugates, which travel downstream towards the test line. Upon reaching the test line, immunocomplex formation initiates and AuNPs remain bound on the test line if the analyte is present. The remaining unbound conjugates are washed away and collected by the absorption pad.

The majority of lateral flow tests require a visual analysis (e.g. a change in colour of the test line) for result interpretation. This can lead to errors as such interpretation is highly subjective. The accuracy of result interpretation can be improved if carried out by a trained professional; however, this puts a limitation on the use of LFTs in settings where trained professionals are not available.

Digital LFTs have been proposed, in which the result of a test is interpreted by an electronic reader. An example is an optical pregnancy test, which makes use of the optical properties of common labels used in visual LFTs such as AuNPs. A fluorescent LFT system has also been proposed, in which a fluorescent tag replaces common labelling molecules such as AuNPs, and the system requires a light source to induce and read the thus generated fluorescence. Further, an electrochemical LFT system has been proposed, in which the presence of labelled immunocomplex is read electrochemically by an electronic reader.

However, unlike enzyme/catalyst-based assays such as ELISA in which the enzymatic/catalytic labels used amplify the signal, the use of non-catalytic labelling molecules such as gold nanoparticles or fluorescent tags in LFTs cannot achieve the same level of sensitivity and specificity. Thus, LFTs generally show a lower level of analytical performance compared to ELISA tests performed in centralised laboratories. To implement an ELISA-type assay on an LFT, a multi-step process is required, but such a multi-step approach is undesirable for a user and errors may be introduced into the test by an untrained user.

The Applicant has recognized that there remains scope for improving the analytical performance of lateral flow tests.

### SUMMARY

In view of the foregoing, an aspect of the present technology provides a device for performing a lateral flow test on a liquid sample, comprising:
a test strip which comprises:
   a membrane having a first capturing reagent disposed on a first surface at a test position, the first capturing reagent being configured to capture an analyte in the liquid sample;
   a sample pad disposed on the first surface at a first end of the membrane spaced apart from the test position, the sample pad being configured to receive the liquid sample; and
   a conjugate pad disposed on the first surface between the sample pad and the test position, the conjugate pad having deposited thereon a catalytic labelling reagent, the catalytic labelling reagent being configured to chemically bind to the analyte;
an electrode array disposed over the membrane configured to apply an electrical potential across the first surface upon activation, the electrode array having deposited thereon at least a component of a catalyst substrate, the catalyst substrate being capable of chemically reacting with the catalytic labelling reagent upon activation; and
a support structure configured, when the device is in an inactive state, to affix the test strip and the electrode array such that the electrode array is separated from the membrane.

According to embodiments of the present technology, the conjugate pad is impregnated with a catalytic labelling reagent, which is dissolved by a liquid sample or sample-buffer mixture as it travels along the membrane. The membrane may for example be a hydrophilic membrane such as a nitrocellulose membrane. The catalytic labelling reagent reacts with the analyte (if present) in the solution forming an immunocomplex, which is captured by the capturing reagent upon the solution reaching the test position. Embodiments of the LFT device is further provided with an electrode array with a catalyst substrate, or at least a component of a catalyst substrate, deposited thereon, e.g. in a dry form, which is separated from the membrane through the support structure before the device is “activated”. Thus, the catalyst substrate is prevented initially from contacting the liquid sample. In doing so, it is possible to prevent the catalytic labelling reagent from reacting with the catalyst substrate while the analyte in the liquid sample forms an immunocomplex with the catalytic labelling reagent and captured by the capturing reagent.

In some embodiments, the catalytic labelling reagent may comprise glucose oxidase, metallic nanoparticles, an enzyme, a nanozyme, a ribozyme.

In some embodiments, the catalytic labelling reagent may comprise glucose oxidase, horseradish peroxidase, alkaline phosphatase, or any enzyme or catalyst that can convert a substrate to a product while not being consumed itself.

In some embodiments, the at least a component of the catalyst substrate may comprise glucose, silver ions, a reducing agent.

In some embodiments, the at least a component of the catalyst substrate may comprise one or more of: glucose; 3,3′,5,5′-tetramethylbenzidine (TMB); 2,2′-azino-di-[3-ethylbenzthiazoline-6-sulfonic acid] (ABTS); 1-napthyl phosphate; L-ascorbic acid phosphate; p-nitrophenyl phosphate, phenol phosphate; hydroquinone diphosphate; silver ions; a reducing agent and similar.

In some embodiments, upon the liquid sample being deposited on the sample pad, the support structure may be configured to facilitate bringing the electrode array into contact with the membrane to activate the device such that contact between the electrode array and the membrane dissolves the at least a component of the catalyst substrate in the liquid sample. The LFT device is “activated” when the electrode array is brought into contact with the membrane soaked with the liquid sample, such that the catalyst substrate, or a component thereof, contacts the liquid sample and is dissolved by the water content of the liquid sample. In doing so, the catalyst substrate is introduced to the liquid sample, in particular the immunocomplex formed by the catalytic labelling reagent and analyte, in a simple step.

There may be many different ways and mechanisms for activating the catalyst substrate, depending on many factors such as the catalytic labelling reagent used, the catalyst substrate used, the analyte to be detected, the desired detection sensitivity and/or specificity, etc. In some embodiments, the electrode array may be configured such that operation of the electrode array oxidises or reduces the at least a component of the catalyst substrate to activate the at least a component of the catalyst substrate whereby the at least a component of the catalyst substrate becomes water-soluble. In doing so, the catalyst substrate is only activated in the liquid sample (soaked into the membrane) when the electrode array is operating. It is therefore possible to reduce or altogether prevent unintentionally activation of the catalyst substrate.

In some embodiments, the at least a component of the catalyst substrate may form the catalyst substrate when dissolved by the liquid sample on the membrane to enable a catalytic reaction between the catalyst substrate and the catalytic labelling reagent.

In some embodiments, the electrode array may be configured to detect a presence of the analyte in the liquid sample by detecting the catalytic reaction between the catalyst substrate and the catalytic labelling reagent. For example, any suitable electrochemical detection techniques may be implemented for the detection of the catalytic reaction between the catalyst substrate and the catalytic labelling reagent by the electrode array, such as by monitoring electrochemical reactions e.g. between the electrode and the catalyst substrate or a produce of the catalytic reaction.

In some embodiments, the electrode array may be configured to detect the catalytic reaction by detecting a reaction substrate, product, or unreacted catalytic labelling reagent, or any combination thereof, e.g. by implementing one or more electrochemical detection techniques.

In some embodiments, the electrode array may further have deposited thereon a catalytic moiety. Herein, a moiety refers to a distinct part or component that forms a larger molecule—in this case a catalyst molecule. Such moiety can also facilitate electron transfer. Such moieties may act as electron mediators, for example, potassium ferrocyanide and methylene blue and similar. The electron mediators may lower the electrode potential required for detection of a specific substance such as catalyst substrate or product.

In some embodiments, the at least a component of the catalyst substrate may form the catalyst substrate when dissolved by the liquid sample on the membrane to enable a catalytic reaction between the catalyst substrate and the catalytic labelling reagent to form a secondary catalyst substrate.

In some embodiments, the secondary catalyst substrate may react catalytically with the catalytic moiety to form a secondary reaction product.

Thus, a two-part catalytic reaction is introduced to the LFT device, which forms a range of products that may be detectable and quantifiable electrochemically for finer-grained result interpretation.

In some embodiments, the electrode array may be configured to detect the secondary reaction product.

In some embodiments, the electrode array may be configured to detect the secondary catalyst substrate.

In some embodiments, the catalytic moiety may comprise an enzyme, a nanoparticle, a nanozyme, or a combination thereof.

In some embodiments, the secondary catalyst substrate may comprise one or more substrates capable of undergoing a redox reaction, optionally such as hydrogen peroxide, 4-aminophenyl phosphate, or o-aminophenol.

In some embodiments, the secondary reaction product may comprise one or more oxidation or reduction products capable of being monitored using an electrochemical technique, optionally such as water or one or more oxidised sugars.

There may be various suitable ways and mechanisms for the support structure to separate the membrane from the electrode array, as long as the electrode array is prevented from contacting the membrane. In some embodiments, the support structure may be configured to spatially separate the membrane from the electrode array.

In some embodiments, the device may comprise an insulating film disposed between the membrane and the electrode array to chemically separate the membrane from the electrode array. In such embodiments, there may or may not be spatial separation between the electrode array and the membrane.

In some embodiments, the insulating film may comprise one or more polymer-based films configured to degrade in a water-based solution, optionally such as a polysaccharide. The one or more polymer-based films may be made/constructed such that it degrades while in contact with the membrane being soaked with the liquid sample as a result, for example, of being dissolved due to prolonged exposure to water, an increase in pressure due to compression by the electrode array onto the membrane, or an electrochemical stimulation due to operation of the electrode array.

In some embodiments, the insulating film may be arranged to be removable to bring the electrode array into contact with the membrane. For example, the support structure may be configured with a mechanism, such as a slider mechanism, to automatically remove the insulating film.

In some embodiments, the insulating film may be configured to dissolve upon contact with liquid.

In some embodiments, the support structure may comprise a compressible element configured to compress the insulating film upon activation. For example, the support structure may comprise elements that, upon compression of the insulating film, pierces the insulating film, or compression of the insulating film may change a physiochemical property of the insulating film, causing it to degrade or disintegrate.

In some embodiments, the electrode array may comprise at least a first pair of corresponding electrodes, the first pair of corresponding electrodes being arranged to overlay the test position on the membrane upon activation, and, optionally, the electrode array comprises a second pair of corresponding electrodes, the second pair of corresponding electrodes being arranged to overlay a background of the membrane.

Another aspect of the present technology provides an electronic reader for reading a result from a lateral flow test device as described above, the electronic reader comprising:
a receiving portion for receiving the lateral flow test device comprising an activation mechanism configured to activate the lateral flow test device; and
a reading port configured to electrically couple with the electrode array of the lateral flow test device to generate an electrical signal in the electrode array and to receive a resulting electrochemical signal through the electrode array,
wherein the activation mechanism of the receiving portion is configured to activate the lateral flow test device by bringing the electrode array into contact with the membrane.

According to embodiments of the present technology, an electronic reader may be provided to electronically read the result of a lateral flow test device as described above. The electronic reader may be configured to read an electrochemical signal generated by a specific substance present in the test strip, such as a product of a catalytic reaction between the catalyst substrate and the catalytic labelling reagent, or in alternative embodiments, the electronic reader may be configured to read an optical signal (e.g. fluorescence) of a substance present in the test strip. The electronic reader is further configured with an activation mechanism for activating the lateral flow test device upon insertion into the electronic reader, to bring the electrode array of the device into contact with the membrane such that the catalyst substrate (or at least a component thereof) may be activated/dissolved through contact with the liquid sample on the membrane.

The activation mechanism may take different forms as desired. In some embodiments, the activation mechanism of the receiving portion may bring the electrode array into contact with the membrane by compressing a portion of the lateral flow test device. For example, the activation mechanism may take the form of a button or lever which, when compressed, physically pushes the electrode array onto the membrane (or vice versa), or it may take the form of a ramp which, when the device is pushed into the reader, reduces the distance between the electrode array and the membrane until they eventually come into contact.

In some embodiments, compressing the portion of the lateral flow test device may reduce a distance between the electrode array and the membrane.

In some embodiments, the electrode array and the membrane may be separated by an insulating film, and compressing the portion of the lateral flow test device pierces through the insulating film. For example, the activation mechanism may comprise sharp protrusions, e.g. pins, spikes, edges, etc., for piercing through the insulating film.

In some embodiments, the electrode array and the membrane may be separated by an insulating film, and the activation mechanism of the receiving portion brings the electrode array into contact with the membrane by peeling off or otherwise removing the insulating film. For example, the activation mechanism may be configured to catch an edge or corner of the insulating film as the device is inserted into the reader, and the action of the insertion peels or pulls the insulating away from the membrane or the electrode array. Alternatively, the insulating film may be made of a material that degrades upon compression or application of pressure, or dissolves upon contact with water.

A further aspect of the present technology provides a method of performing a lateral flow test on a liquid sample using a lateral flow test device, the device comprising:
a test strip which comprises:
   a membrane having a first capturing reagent disposed on a first surface at a test position, the first capturing reagent being configured to capture an analyte in the liquid sample;
   a sample pad disposed on the first surface at a first end of the membrane spaced apart from the test position, the sample pad being configured to receive the liquid sample;
   a conjugate pad disposed on the first surface between the sample pad and the test position, the conjugate pad having deposited thereon a catalytic labelling reagent, the catalytic labelling reagent being configured to chemically bind to the analyte;
an electrode array disposed over the membrane configured to apply an electrical potential across the first surface upon activation, the electrode array having deposited thereon at least a component of a catalyst substrate, the catalyst substrate being capable of chemically reacting with the catalytic labelling reagent upon activation; and
a support structure configured, when the device is in an inactive state, to affix the test strip and the electrode array such that the electrode array is separated from the membrane, and the method comprising:
   depositing the liquid sample onto the sample pad of the test strip;
   bringing the electrode array into contact with the membrane to activate the lateral flow test device;
   operating the electrode array to apply an electrical potential across the first surface through the electrode array to drive an electrochemical reaction in the test strip; and
   measuring a resulting electrochemical signal from the test strip through the electrode array to detect a presence of the analyte in the liquid sample.

According to embodiments of the present technology, since the electrode array of the lateral flow test device has deposited thereon a catalyst substrate, or at least a component of the catalyst substrate, e.g. in a dry form, activation of the lateral flow test device to commence a catalytic reaction between the catalyst substrate and the catalytic labelling reagent on the membrane may be achieved in a single step simply by bringing the electrode array into contact with the membrane, after a liquid sample has been introduced onto the sample pad.

In some embodiments, bringing the electrode array into contact with the membrane may cause the at least a component of the catalyst substrate to be dissolved by the liquid sample on the membrane.

In some embodiments, operating the electrode array may oxidise or reduce the at least a component of the catalyst substrate to activate the at least a component of the catalyst substrate whereby the at least a component of the catalyst substrate becomes water-soluble.

In some embodiments, the at least a component of the catalyst substrate may form the catalyst substrate when dissolved by the liquid sample on the membrane to enable a catalytic reaction between the catalyst substrate and the catalytic labelling reagent.

In some embodiments, measuring a resulting electrochemical signal from the test strip through the electrode array may comprise measuring the catalytic reaction between the catalyst substrate and the catalytic labelling reagent. For example, any suitable electrochemical detection techniques may be implemented for the detection of the catalytic reaction between the catalyst substrate and the catalytic labelling reagent by the electrode array, such as by monitoring electrochemical reactions e.g. between the electrode and the catalyst substrate or a produce of the catalytic reaction.

In some embodiments, measuring the catalytic reaction between the catalyst substrate and the catalytic labelling reagent may comprise measuring an amount of a reaction product, or an amount of unreacted catalytic labelling reagent, or both, e.g. by implementing one or more electrochemical detection techniques.

In some embodiments, the electrode array may further have deposited thereon a catalytic moiety.

In some embodiments, the at least a component of the catalyst substrate may form the catalyst substrate when dissolved by the liquid sample on the membrane to enable a catalytic reaction between the catalyst substrate and the catalytic labelling reagent to form a secondary catalyst substrate.

In some embodiments, the secondary catalyst substrate may react catalytically with the catalytic moiety to form a secondary reaction product.

In some embodiments, measuring a resulting electrochemical signal from the test strip through the electrode array may comprise measuring an amount of the secondary reaction product.

In some embodiments, measuring a resulting electrochemical signal from the test strip through the electrode array may comprise measuring an amount of the secondary catalyst substrate.

In some embodiments, measuring a resulting electrochemical signal from the test strip through the electrode array may comprise potentiometry, amperometry, voltammetry, one or more impedance-based measurements, one or more capacitive measurements, or a combination thereof.

A yet further aspect of the present technology provides a device for performing a lateral flow test on a liquid sample, comprising:
a test strip which comprises:
   a membrane having a first capturing reagent disposed on a first surface at a test position, the first capturing reagent being configured to capture an analyte in the liquid sample;
   a sample pad disposed on the first surface at a first end of the membrane spaced apart from the test position, the sample pad being configured to receive the liquid sample; and
   a conjugate pad disposed on the first surface between the sample pad and the test position, the conjugate pad having deposited thereon a catalytic labelling reagent, the catalytic labelling reagent being configured to chemically bind to the analyte;
an activation layer disposed over the membrane arranged to contact the first surface upon activation, the activation layer having deposited thereon at least a component of a catalyst substrate, the catalyst substrate being capable of chemically reacting with the catalytic labelling reagent upon activation; and
a support structure configured, when the device is in an inactive state, to affix the test strip and the activation layer such that the activation layer is separated from the membrane.

According to embodiments of the present technology, the catalytic labelling reagent immobilised on the conjugate pad is dissolved when a liquid sample or sample-buffer mixture is introduced to the sample pad and travels along the membrane. The catalytic labelling reagent reacts with the analyte (if present) in the solution forming an immunocomplex, which is captured by the capturing reagent upon the solution reaching the test position. Embodiments of the LFT device is further provided with an activation layer that has deposited thereon a catalyst substrate, or at least a component of a catalyst substrate, e.g. in a dry form. The activation layer is separated from the membrane, through the support structure, before the device is “activated”. Thus, the catalyst substrate is prevented initially from contacting the liquid sample. In doing so, it is possible to prevent the catalytic labelling reagent from reacting with the catalyst substrate while the analyte in the liquid sample forms an immunocomplex with the catalytic labelling reagent and captured by the capturing reagent.

There may be many ways of implementing the activation layer. In some embodiments, the activation layer may comprise an electrode array configured to apply an electrical potential across the first surface upon activation, and the catalyst substrate, upon reacting with the catalytic labelling reagent, produces an electrochemically detectable product.

In some embodiments, the activation layer may be a substantially transparent foil, and the catalyst substrate, upon reacting with the catalytic labelling reagent, produces a visually detectable product.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

Embodiments will now be described, with reference to the accompanying drawings, in which:

FIG. 1A shows a cycle of chemical reactions in an exemplary catalytic immunosensor;

FIG. 1B illustrate the reaction cycle of FIG. 1A being interrupted;

FIG. 2A shows a first implementation example of a lateral flow test device before activation;

FIG. 2B shows the lateral flow test device of FIG. 2A after activation;

FIG. 3A shows a second implementation example of a lateral flow test device before activation;

FIG. 3B shows the lateral flow test device of FIG. 3A after activation;

FIG. 4A shows a third implementation example of a lateral flow test device before activation;

FIG. 4B shows the lateral flow test device of FIG. 4A after activation;

FIG. 5A shows a fourth implementation example of a lateral flow test device before activation;

FIG. 5B shows the lateral flow test device of FIG. 5A after activation;

FIG. 6A shows an implementation example of a lateral flow test device;

FIG. 6B shows an implementation example of an electronic reader; and

FIG. 7 shows a flow diagram of an exemplary method of operating a lateral flow test device;

FIG. 8 shows component parts of a lateral flow test cassette;

FIG. 9 shows graphically raw data output from a cassette such as that shown in FIG. 8; and

FIG. 10 shows graphically data output from 35 sensors such as that shown in FIG. 8 as exposed to increasing levels of human CRP.

### DETAILED DESCRIPTION

In a lateral flow test using an immunosensor analytical device, the following interactions between a user interacting and the analytical device are typically expected:
1. Opening a kit containing the analytical device (lateral flow test device/cassette) and engaging with a user manual or app that guides the user through the test;
2. Taking a sample, such as blood, blood plasma, blood serum, saliva, urine, swab, stool, as well as other non-biological sample such as water or milk;
3. Unpackaging a sealed packet containing the lateral flow test device (e.g. sealed against contamination) and introducing the sample to the sample pad of the LFT device;
4. Waiting for a predetermined length of time (e.g. as stated in the user manual) for the sample to flow through the membrane of the LFT device;
5. Reading the result of the test;
6. Reporting the Result (if Applicable); and
7. Disposing the Test.

In the case of a catalytic immunosensor, a wash step and a step of introducing a catalyst substrate are required between steps 3 and 4 above, which is burdensome for the user and increases a risk of introducing errors into test.

The present disclosure describes a sensor architecture that allows the introduction of the substrate to be performed during, or as a part of, the result reading step (step 5), thus simplifying the user's interaction with the analytical device and thereby reduces the risk of errors.

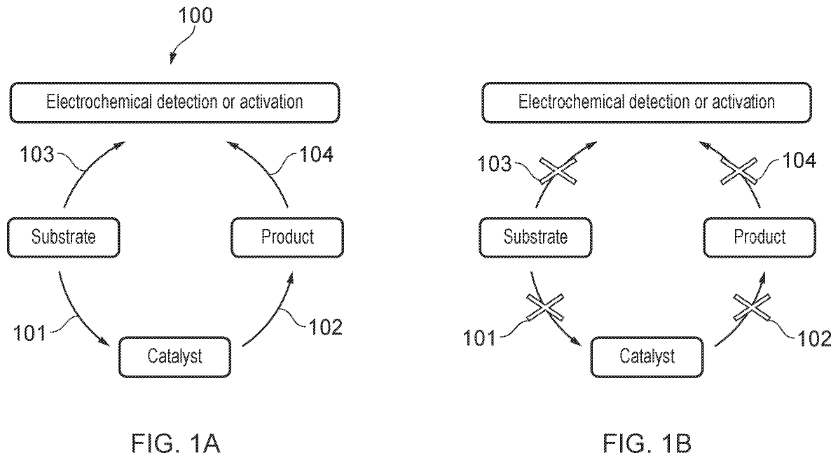

The general principle of a lateral flow test according to the present technology is to interrupt the cycle of chemical reaction that take place around the sample introduction step (step 3) and the sample flowthrough step (step 4), prior to the result reading step (step 5). This principle is illustrated in FIGS. 1A and 1B.

FIG. 1A shows an exemplary cycle 100 of chemical reaction between a catalyst and a corresponding catalyst substrate in a lateral flow test (LFT), e.g. on a membrane of a test strip of the LFT. Introduction of the substrate to the LFT initiates a chemical reaction 101 between the substrate and the catalyst, resulting in the generation 102 of a reaction product. In some embodiments, the substrate may require activation 103 through oxidation or reduction e.g. facilitated by operation of an electrode array. The reaction product generated by the catalytic reaction may be detected 104 electrochemically e.g. by the electrode array.

FIG. 1B illustrates the reaction cycle 100 of FIG. 1A being interrupted. The reaction cycle may be interrupted at any one or more of the stages 101, 102, 103 and 104 and the cycle is subsequently resume to completion during the result reading stage (step 5). For example, the introduction of the substrate stage 101 may be interrupted e.g. by physically separating the substrate from the catalyst, the reaction product generation stage 102 may be interrupted e.g. by introduction of an activation step for the catalyst or substrate, or introduction of a secondary reaction, the substrate activation stage 103 or the reaction product detection stage 104 may be interrupted e.g. not operating the electrode array. In the initial stages of the test procedure (between steps 3 and 4), the reaction cannot proceed due to reaction components necessary to complete the reaction cycle being de-coupled in one or more of the stages 101 to 104, such as through the physical separation of the substrate from the catalyst. At least one component required to complete the reaction cycle is de-coupled from other components so the reaction cannot proceed. Once the reaction components are coupled, and the reaction cycle is completed, the reaction between the substrate and the catalyst to generate a reaction product can proceed.

In existing approaches, such an interruption of the reaction cycle is not necessary as a separate wash step and substrate introduction step must be performed between steps 3 and 4 before the cycle can complete.

The present technology provides a device for performing a lateral flow test on a liquid sample which, by virtue of the construction of the device, facilitates the interruption of the reaction cycle 100 while keeping the substrate and the catalyst both within the same LFT device/cassette, thereby negating the need to introduce the substrate to the LFT device. An LFT device according to the present technology generally comprises a test strip. The test strip comprises a membrane having a capturing reagent disposed on a membrane surface at a test position. The capturing reagent is configured to capture an analyte in the liquid sample. A sample pad is disposed on the membrane surface at a position near one end of the membrane and spaced apart from the test position. The sample pad is configured to receive the liquid sample. A conjugate pad is disposed on the membrane surface between the sample pad and the test position. The conjugate pad has deposited thereon, or impregnated with, a catalytic labelling reagent. The catalytic labelling reagent is configured to chemically bind to the analyte. The LFT device further comprises an activation layer disposed over the membrane, which, upon activation of the LFT device, is arranged to contact the membrane surface. The activation layer has deposited thereon at least a component of a catalyst substrate, for example in its dry form. Upon activation of the LFT device, the catalyst substrate chemically reacts with the catalytic labelling reagent. A support structure is provided to the LFT device, which is configured, when the device is in an inactive state, to affix the test strip and the activation layer in such a way that the activation layer is separated from the membrane. Such separation may for example be a physical separation by a predetermined distance such that the activation layer is spaced apart from the membrane, or a separation by a barrier, e.g. a chemical or moisture barrier, between the activation layer and the membrane, for example by attaching an insulating film over the membrane or over the activation layer, or both. Activation of the LFT device may be regarded as bringing the activation layer into contact with the membrane, whether physically, chemically, or otherwise, for example through reducing the physical distance between the activation layer and the membrane until touching, or through removing a chemical or moisture barrier.

According to embodiments of the present technology, when a liquid sample or sample-buffer mixture is introduced onto the sample pad, the catalytic labelling reagent on the conjugate pad is dissolved by the water content in the liquid sample or sample-buffer mixture as it travels along the membrane. A suitable membrane may for example be a hydrophilic membrane such as a nitrocellulose membrane. The catalytic labelling reagent reacts with the analyte (if present) in the solution to form an immunocomplex, which is captured by the capturing reagent upon the solution reaching the test position. Embodiments of the LFT device is further provided with an activation layer, which is separated from the membrane through the support structure before the device is “activated”. Such an activation layer may for example be a substantially transparent foil or film impregnated with, or has deposited thereon, a catalyst substrate which produces a visually detectable product when reacting with the catalytic labelling reagent, or an electrode array, configured to apply an electrical potential across the membrane surface when operated, which has deposited thereon a catalyst substrate (e.g. in a dry form) that produces an electrochemically detectable product when reacting with the catalytic labelling reagent. Thus, the catalyst substrate is prevented initially from contacting the liquid sample. In doing so, it is possible to prevent the catalytic labelling reagent from reacting with the catalyst substrate while the analyte in the liquid sample forms an immunocomplex with the catalytic labelling reagent and captured by the capturing reagent.

According to embodiments of the present technology, an electronic reader can be provided to electronically read the results of a lateral flow test device as described above. The electronic reader may be configured to read an electrochemical signal generated, e.g. through application of an electrical potential across the membrane by an electrode array, by a specific substance present in the test strip, for example a product of a catalytic reaction between the catalyst substrate and the catalytic labelling reagent. Alternatively, in some embodiments, the electronic reader may be configured to read an optical signal (e.g. fluorescence) of a substance present in the test strip. The electronic reader is further configured with an activation mechanism that activates the LFT device upon insertion into the electronic reader, to bring the activation layer, e.g. an electrode array, of the device into contact with the membrane such that the catalyst substrate (or at least a component thereof) may be dissolved or otherwise activated through contact with the liquid sample on the membrane.

According to embodiments of the present technology, a catalyst substrate is deposited, e.g. in a dry form, on an activation layer (e.g. an electrode array) and separated from a catalytic labelling reagent (comprising the catalyst that reacts with the catalyst substrate) which is deposited on the membrane of the test strip of an LFT device by a support structure. In doing so, the cycle of chemical reaction between the catalyst and the substrate (e.g. the reaction cycle 100) is interrupted. Activation of the LFT device to bring the activation layer into contact with the membrane after introduction of a liquid sample to the membrane completes the reaction cycle by bringing the catalyst into contact with the substrate in a solution, and completion of the reaction cycle causes a catalytic reaction to commence between the catalyst substrate and the catalytic labelling reagent on the membrane. Thus, completion of the reaction cycle can be achieved, according to embodiments of the present technology, in a single step by simply bringing the activation layer (e.g. an electrode array) into contact with the liquid-sample-soaked membrane.

In an implementation example, embodiments of the present technology provide a single-step electrochemical lateral flow test device/cassette, in which a catalyst-mAb (monoclonal antibody) conjugate and a catalyst substrate are immobilised on two separate platforms—the test strip (membrane) and an electrode array (or an electrode of the electrode array). Materials may be selected such that the assembly of the test strip and electrode array is flexible. The two-platform device facilitates the de-coupling of reaction components during sample flowthrough in a lateral flow assay, but upon activation allows the chemical reaction cycle between catalyst and substrate to complete and thereby enable electrochemical sample detection on the same platform.

Alternative embodiments may implement the same principle for visual result interpretation, by replacing the electrode with a transparent feature (e.g. a transparent foil), on which there is immobilized a particulate that promotes colour, or any other visual signal, development after the reaction cycle is coupled.

The substrate can be a single substrate, which can be immobilised on the conjugate pad, electrode array or be present in a liquid sample (either in a buffer in which a sample is introduced or the sample itself). In this scenario, the reaction is solely controlled by the presence of the substrate. In an example, if the substrate is the analyte that is the subject of the test, the catalyst-substrate reaction can be used to determine an analyte concentration. In some embodiments, a reaction may require two or more substrates, and the reaction cycle may be interrupted through absence of one of the substrates before the result reading step (step 5). In some embodiments, the substrate may be in an inactive form, for example a dry form, that requires it to be mixed with a solution for it to convert into its active form; thereafter the activated (e.g. dissolved) substrate may participate in the desired catalyst-substrate reaction. In other embodiments, electrochemical oxidation or reduction may be employed to convert a non-reactive (inactive) substrate into a reactive (active) substrate to complete the reaction cycle. In further embodiments, a plurality of substrates may be introduced through immobilisation of the plurality of substrates on a single platform. For example, in a first (inactive) state, silver ions and a reducing agent are both in a non-active form and are co-immobilised on a working electrode (activation layer) using a low pH solution which ensures preservation of the inactivated state. Upon activation, both the silver ions and inactive reducing agent dissolve in the membrane. In the present example, dissolution in a running buffer with a higher pH than the reducing agent causes the reducing agent to activate, allowing silver enhancement to occur on an AuNP (gold nanoparticle) catalyst. In the present example, electrochemical measurement may be performed based on e.g. a detection of freely available silver ions in the vicinity of the electrode.

The catalyst used in embodiments of the present technology may be any suitable catalyst. For example, the catalyst may be a biological catalyst (e.g. an enzyme), a metallic catalyst (e.g. a metal nanoparticle), a nanozyme, a ribozyme, or any particulate capable of converting a corresponding substrate to a product, or lowering an activation energy sufficiently for reaction conversion without itself being consumed. In another embodiment, the catalyst may be replaced with a liposome or any other encapsulating agent, which is disrupted upon reaction cycle completion and releases either a catalyst or an electrochemically detectable molecule.

The reaction product may be an electrochemically active product that can be detected using electrochemical techniques, a visually or optically detectable product, or a mediator capable of enhancing a detection of a different product generated in the buffer, electrode or the conjugate pad. The same product may act as a substrate for a secondary reaction that may e.g. be detected electrochemically.

As described above, the separation of the reaction components may either be spatial (physically spaced apart) that requires movement to couple the components, or through the use of a barrier such as an insulating layer or film that prevents contact of the components. Such an insulating layer may be pressure-sensitive that is activated through application of pressure/compression, or it may be physically removed or peeled off by a user or an automated mechanism prior to the test read. In other examples, such an insulating layer may be made of a dissolvable material that is in contact the membrane during sample flowthrough, and slowly dissolves over time to facilitate a time-dependent activation of the reaction. Other forms of reaction components separation to interrupt the reaction cycle prior to the result reading step and subsequent re-coupling of the reaction components during have been contemplated.

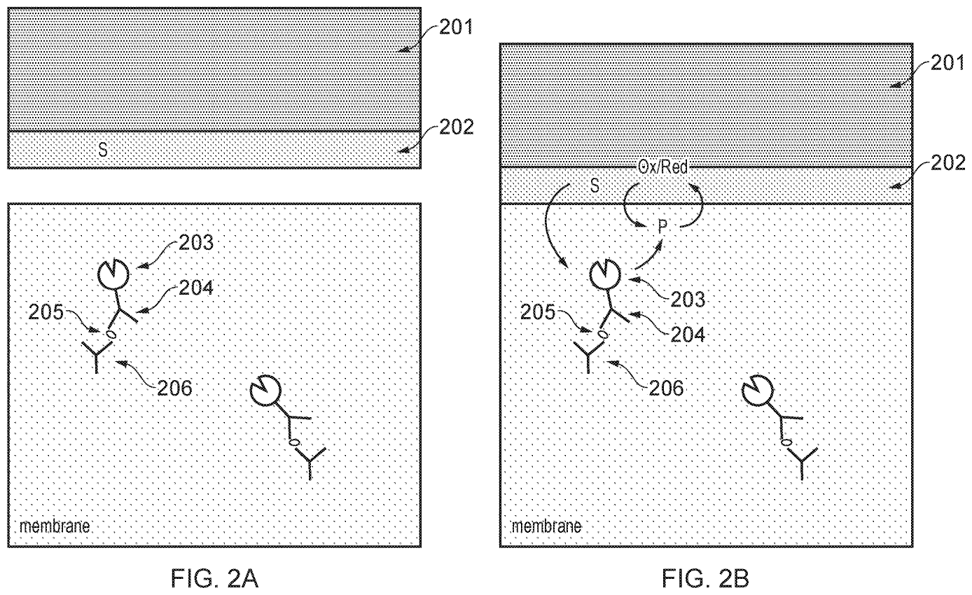

FIG. 2A shows a first implementation example of a lateral flow test device before activation, according to an embodiment. The device of the first example comprises an electrode 201 having deposited/immobilized thereon a substrate 202 (e.g. glucose-), catalytic labelling molecules 203 are immobilized on the membrane (e.g. glucose oxidase in a conjugate pad), which is conjugated to detect mAb 204 that forms an immunocomplex with an analyte 205 and capturing mAb 206. To ensure no contact occurs before measurement, the catalyst 203 is immobilized on the membrane while the substrate 202 is immobilized on the electrode surface 201 in a dried form. Upon application of a sample to the device (e.g. on a sample pad), capillary flow occurs allowing resuspension of the catalyst-mAb conjugate, and immunocomplex formation (with catalytic label) at the test line (if the antigen is present) and control line before termination at an absorption pad. When result reading and/or analysis is required, the device is activated whereby the electrode 201 (e.g. as part of an array) is brought into contact with one or more areas (e.g., test line, control line or background) of the membrane. Upon contact, the substrate 202 on the electrode 201 dissolves on the membrane with a reaction that only occurs if the immunocomplex with the catalytic labelling molecule 203 is present.

Importantly, since the electrode is in contact with the membrane, the reaction can be monitored electrochemically for example by measuring a rate of substrate depletion or a rate of product generation.

FIG. 2B shows the lateral flow test device of FIG. 2A after activation. Upon device activation, contact between the electrode 201 and the membrane causes dissolution of the substrate 202 in the proximity of the catalyst 203. Once the device transitions to an activated state and the electrode 201 comes into contact with the membrane, the substrate 202 is able to dissolve in the membrane, where it is consumed by the catalytic labelling molecule 203. The depletion of the substrate 202 or the formation of a reaction product may then be measured electrochemically as a consequence of the catalytic reaction. For example, the reaction product formed during the catalytic reaction may subsequently be monitored in-situ in accordance with its local environment through electrochemical oxidation or reduction of the product. Multiple electrochemical methods may be used including (but not limited to) potentiometry, amperometry, voltammetry, impedance-based measurements, and capacitive measurements.

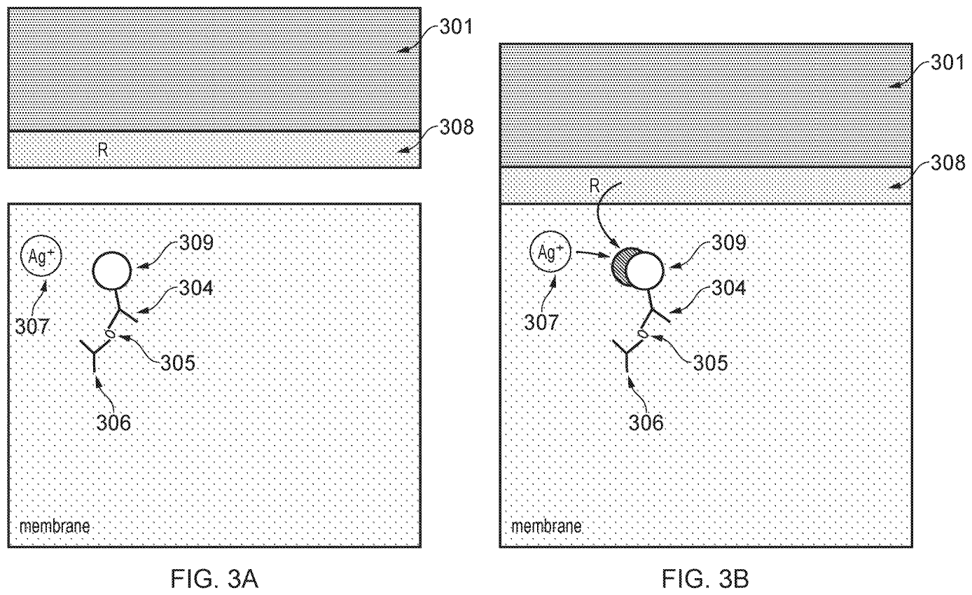

In an example, a metal nanoparticle may serve as a catalytic label such as a gold nanoparticle, which is conjugated to an antibody. Embodiments of the present technology is compatible with a silver enhancement mechanism, whereby silver ions (serving as a substrate) are reduced on the surface of gold nanoparticles AuNP (serving as a catalyst) with the aid of a reducing agent (an activator), as illustrated in FIGS. 3A and 3B.

FIG. 3A shows a second implementation example of a lateral flow test device before activation, according to an embodiment. Silver ions 307 may be present in a buffer or deposited on the conjugate or sample pad, while a reducing agent 308 may be immobilized on an electrode surface 301. In an inactive state, as shown in FIG. 3A, silver ions 307 are present in a running buffer and therefore do not react with AuNPs 309 during a sample flowthrough or the subsequent capturing of the AuNPs 309 on the membrane, by detect mAb 304 that forms an immunocomplex with an analyte 305 and capturing mAb 306. In the present example, the reducing agent 308 is immobilized on the electrode 301 and is physically separated from the membrane, preventing a reaction between catalyst and substrate from occurring.

In an activated stage, as shown in FIG. 3B, the reaction between catalyst and substrate is activated by an introduction of the reducing agent 308 into the membrane. As the reducing agent 308 is introduced, the silver ions (Ag+) 307 are reduced specifically on the surface of the AuNPs 309 due to its catalytic properties. This reaction may be electrochemically monitored by measurement of the available free silver ions in the electrode proximity. In the present example, AuNPs 309 only react when the reducing agent 308 is introduced to the membrane.

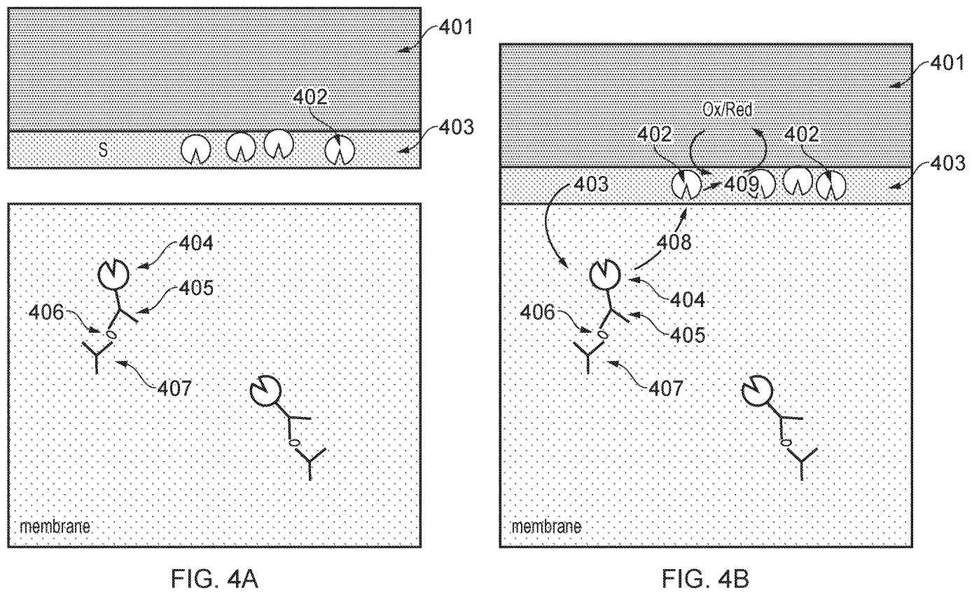

Another embodiment may involve an electrode coated with a combination of a catalytic moiety and a substrate, as shown in FIGS. 4A and 4B.

FIG. 4A shows a third implementation example of a lateral flow test device before activation, according to an embodiment. In an inactive state, an electrode array 401 separated from the membrane. The electrode array 401 has immobilized thereon a catalytic moiety 402 (e.g. an enzyme, nanoparticle, or nanozyme, etc.) and a substrate 403 co-immobilized on the electrode surface 401 alongside the catalytic moiety 402. In the present example, the substrate 403 can only be catalyzed by a catalyst 404 in the membrane but not by the catalytic moiety 402 on the electrode 401. The catalyst 404 on the membrane is conjugated to detection mAb (labelling reagent) 405, which is capable of forming an immunocomplex with an analyte 406 and capturing mAb (capturing reagent) 407.

FIG. 4B shows the device of FIG. 4A in an activated state when the electrode 401 is in contact with the membrane. Upon activation, the substrate 403 and catalytic moiety 402 are delivered to the membrane through contact with the electrode 401. The substrate 403 is dissolved in the membrane by the water content in a liquid sample, and the catalyst 404 consumes the dissolved substrate 403 in a catalytic reaction to form a first product 408. The first product 408 in turn acts as a substrate for the catalytic moiety 402, now present in the membrane, allowing the formation of a second product 409. In the present example, any one or more of the substrate 403, the first product 408, the second product 409, the catalytic moiety 402 or the catalyst 404 may be monitored electrochemically to enable the determination of immunocomplex quantity, which, in the case of the test line, is in turn directly related to analyte concentration.

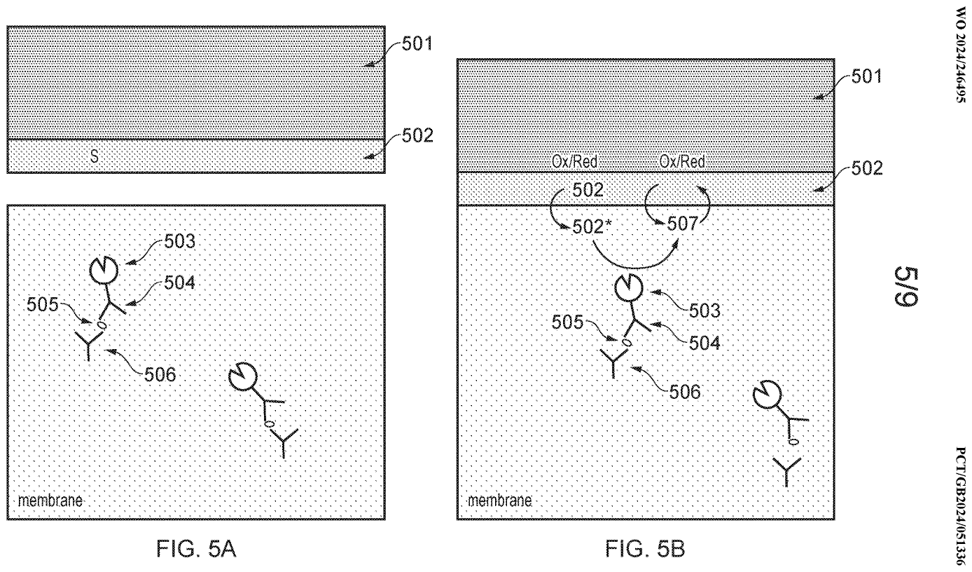

In a further alternative embodiment, a substrate may transition from an inactive state to an active state, as shown in FIGS. 5A and 5B.

FIG. 5A shows a fourth implementation example of a lateral flow test device in an inactive state, according to an embodiment, in which an electrode 501 is spatially separated from a membrane of the LFT device. The working electrode 501 has deposited thereon a substrate 502 in an inactive state. Upon sample introduction, an immunocomplex forms in the membrane between a catalytic moiety 503 and detection antibody (labelling reagent) 504 conjugate, an antigen 505 and a capture antibody (capturing reagent) 506.

In FIG. 5B, device activation brings into contact the electrode 501 and the membrane to promote a subsequent catalytic reaction. In the present example, the electrode 501 activates the substrate 502 to an activated state 502* through oxidation or reduction when the electrode 501 is in operation. The substrate 502 is selected for its property that the substrate state is only soluble, e.g. in the liquid-soaked membrane, in its activated state 502*. The activated substrate 502* can then be converted to a measurable product 507 through catalysis by the catalytic moiety 503 and detection antibody 504 conjugate.

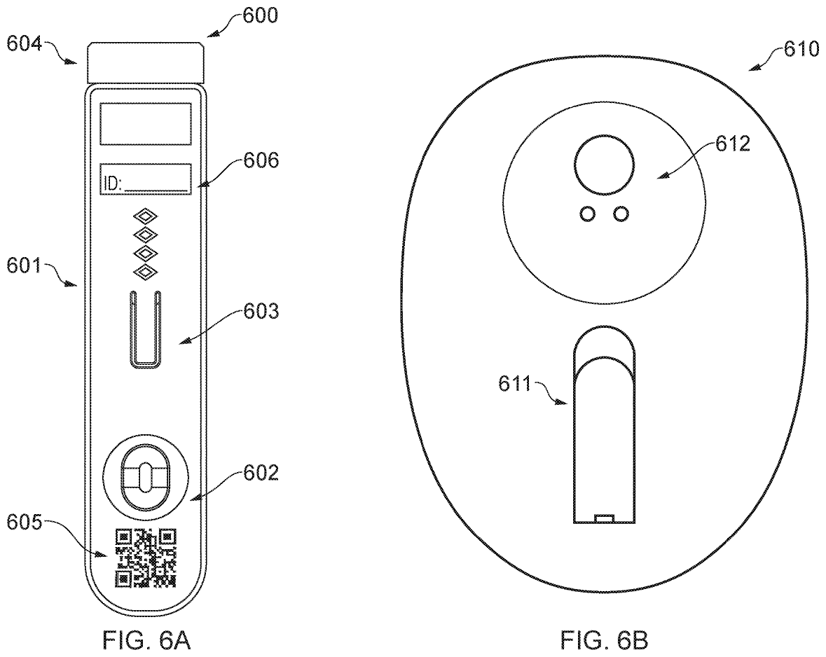

FIG. 6A shows an implementation example of a lateral flow test device 600, according to an embodiment. The LFT device 600 comprises a housing 601 formed of a material, e.g. plastic, capable of protecting a test strip (not shown) housed within. The device is provided with an opening 602 for access to a sample pad of the test strip for introduction of a sample. The housing 601 may be configured with a mechanism such as a lever 603 that can be compressed to bring an electrode into contact with the test strip to activate the device 601. The result of the test may be read electronically through an electronic port 604. Moreover, the device 600 may be provided with a unique identification code 605 for identifying the device 600, and a labelling area 606 to label the device 600 as desired.

FIG. 6B shows an implementation example of an electronic reader 610, according to an embodiment. The electronic reader 610 may be used for reading the result of an LFT device, such as the device 600, through an electronic port, such as the electronic port 604. The electronic reader 610 in the present example is provided with a compression mechanism 611 for compressing (to apply a pressure) a corresponding compressible portion 603 of the device 600. A button 612 or other activation mechanism may be provided to the electronic reader 610 to commence a result reading.

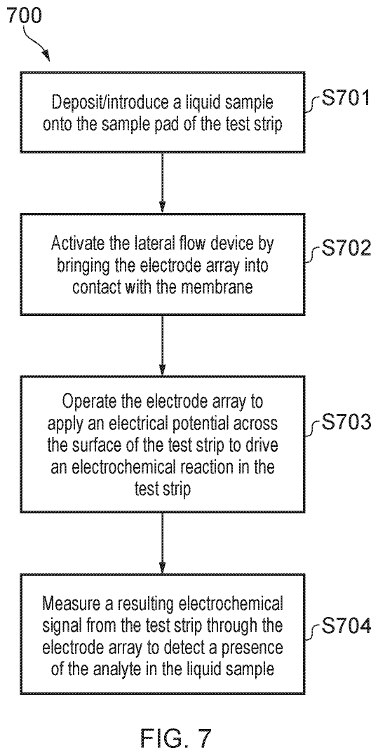

FIG. 7 shows a flow diagram of an exemplary method 700 of operating a lateral flow test device. The method 700 of performing a lateral flow test on a liquid sample may use a lateral flow test device such as the device 600 and embodiments thereof described with reference to FIGS. 2 to 5. Such a LFT device generally comprises a test strip, an electrode array (activation layer) and a support structure. The test strip generally comprises a membrane having a capturing reagent disposed on a first surface at a test position, the capturing reagent being configured to capture an analyte in the liquid sample, a sample pad disposed on the membrane surface at one end of the membrane spaced apart from the test position, the sample pad being configured to receive the liquid sample, a conjugate pad disposed on the membrane surface between the sample pad and the test position, the conjugate pad having deposited thereon a catalytic labelling reagent, the catalytic labelling reagent being configured to chemically bind to the analyte. The electrode array may be disposed over the membrane and is generally configured to apply an electrical potential across the membrane surface upon activation, the electrode array having deposited thereon at least a component of a catalyst substrate, the catalyst substrate being capable of chemically reacting with the catalytic labelling reagent upon activation. The support structure is generally configured, when the device is in an inactive state, to affix the test strip and the electrode array such that the electrode array is separated from the membrane.

In the present example, the method 700 begins with S701 in which the liquid sample is deposited or otherwise introduced onto the sample pad of the test strip. Then at S702, the electrode array is brought into contact with the membrane to activate the lateral flow test device. Thereafter, at S703, the electrode array is operated to apply an electrical potential across the membrane surface through the electrode array to drive an electrochemical reaction in the test strip. At S704, a resulting electrochemical signal is measured from the test strip through the electrode array to detect a presence of the analyte in the liquid sample.

Further details regarding some methods of sensor manufacture are set out below.

### Methodology

Lateral flow strips are manufacturable using typical manufacturing processes. In particular, according to one method, capturing mouse anti-human CRP antibodies (1 mg/mL) were deposited on a nitrocellulose membrane and dried at 37 °C. A detection antibody was conjugated to alkaline phosphatase using an off-the shelf conjugation kit and dried in conjugate pads composed of glass fiber. Sample and wicking pads were laminated on to the plastic backing cards, which were then cut into strips to complete strip manufacture.

Electrodes are manufactured according to typical electrode manufacturing processes. In one example, FR4 forms the basis of the electrode where the traces are outlined in a traditional printed circuit board manufacture process involving photolithography and metal plating. The working electrode can then be screen printed using carbon paste to create electrochemical surfaces of interest. Ag/AgCl ink can be used to create reference electrodes. Working electrodes can be functionalized using a functionalization solution which includes water, ascorbic acid phosphate, 150 mM tris-acetate buffer and a binder such as sucrose or hydroxy ethyl cellulose. This can be deposited on the electrode using automated dispensing, and then dried.

In one example, LFT cassettes can be injection molded and designed to be shaped to carry and support the LFT strip and the electrodes.

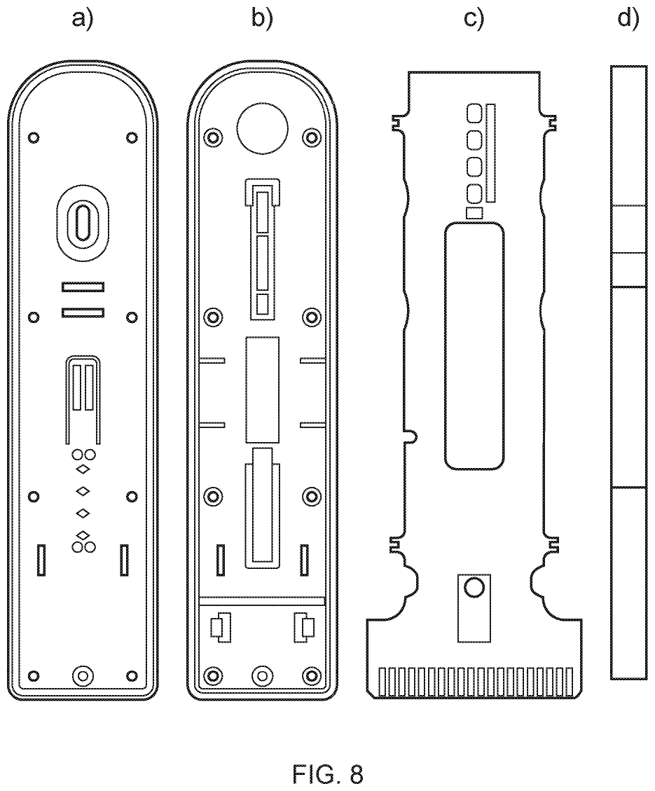

FIG. 8 shows component parts of a lateral flow test cassette. FIG. 8a shows a lid or “top” part of a cassette. FIG. 8b shows a bottom or “base” part of a cassette. FIG. 8c shows an electrode array with a catalyst substrate. FIG. 8d shows a lateral flow test strip having a catalytic label.

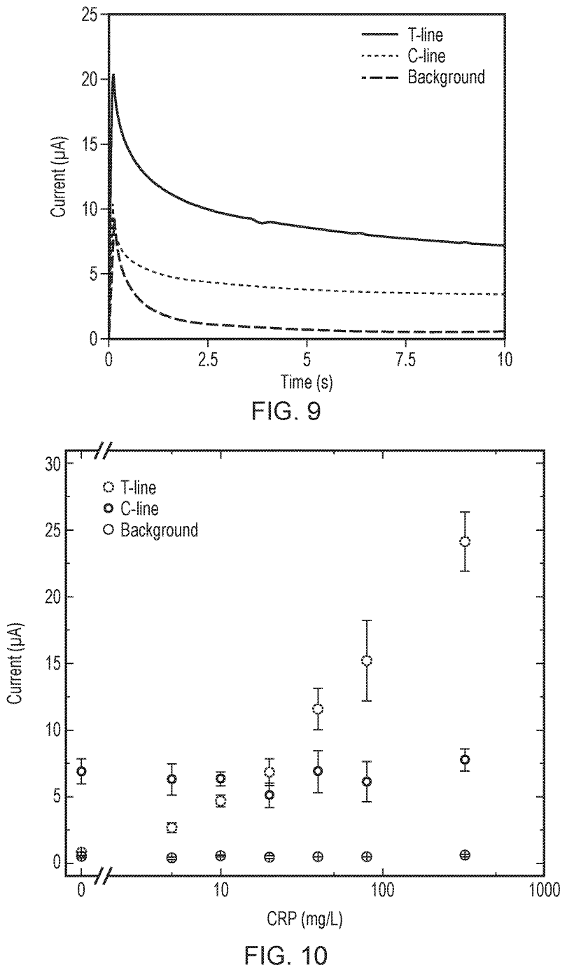

FIG. 9 shows graphically raw data output from a cassette such as that shown in FIG. 8. In one example, device testing was performed by using a 150 mM Tris-acetate buffer supplemented with 1% Tween 20 and 0.05% Proclin, where a sample (CRP depleted human serum, spiked with recombinant CRP) was mixed with the running buffer in the ratio 1:100, before 70 μL were dropped on the LFT and flown for 10 min. Measurement of current was made after inserting the cassette into the reader, which allowed the electrode array to touch the LFT. The electrochemical measurement is chronoamperometry set at 0.3 V vs. the reference electrode for a duration of 10 seconds on each electrode. Data was recorded in relation to the current produced before the test line (background), on the test line and on the control line. The raw data output shown in FIG. 9 relates to a system such as that shown (in pieces) in FIG. 8. FIG. 9 outlines a current response from a sample supplemented with 40 mg/L of CRP. It can be seen from FIG. 9 that electrode positioned before the T-line (background electrode) exhibits low current, the electrode overlaying the test line exhibits high current and the electrode overlaying the control line exhibits lower current than the test line, and higher than the background.

FIG. 10 shows graphically data output from 35 sensors such as that shown in FIG. 8 as exposed to increasing levels of human CRP, in five repeats. In the example shown in FIG. 10, increasing CRP levels were tested covering a range of clinically relevant CRP concentrations. FIG. 10 illustrates graphically data output from 35 sensors exposed to increasing concentrations of human CRP in CRP-free serum. In a sample where there is no CRP (blank sample), the background and test line currents are low, but the control line current are significantly higher. Such a result demonstrates that the test was executed appropriately. As the concentration of CRP increases, the T-line current increases, while the background and control line current remain stable. Such a result demonstrates that the sensors respond specifically to the level of CRP in a sample.

The examples and conditional language recited herein are intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its scope as defined by the appended claims.

Furthermore, as an aid to understanding, the above description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to limit the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

It will be clear to one skilled in the art that many improvements and modifications can be made to the foregoing exemplary embodiments without departing from the scope of the present techniques.

1-43. (canceled)

44. A device for performing a lateral flow test on a liquid sample, comprising:
a test strip which comprises:
a membrane having a first capturing reagent disposed on a first surface at a test position, the first capturing reagent being configured to capture an analyte in the liquid sample;
a sample pad disposed on the first surface at a first end of the membrane spaced apart from the test position, the sample pad being configured to receive the liquid sample; and
a conjugate pad disposed on the first surface between the sample pad and the test position, the conjugate pad having deposited thereon a catalytic labelling reagent, the catalytic labelling reagent being configured to chemically bind to the analyte;
an electrode array disposed over the membrane configured to apply an electrical potential across the first surface upon activation, the electrode array having deposited thereon at least a component of a catalyst substrate, the catalyst substrate being capable of chemically reacting with the catalytic labelling reagent upon activation; and
a support structure configured, when the device is in an inactive state, to affix the test strip and the electrode array such that the electrode array is separated from the membrane.

45. The device of claim 44, wherein the catalytic labelling reagent comprises glucose oxidase, metallic nanoparticles, an enzyme, a nanozyme, a ribozyme, or combination thereof.

46. The device of claim wherein the at least a component of the catalyst substrate comprises glucose, silver ions, a reducing agent.

47. The device of claim 44, wherein upon the liquid sample being deposited on the sample pad, the support structure is configured to facilitate bringing the electrode array into contact with the membrane to activate the device such that contact between the electrode array and the membrane dissolves the at least a component of the catalyst substrate in the liquid sample.

48. The device of claim 47, wherein the electrode array is configured such that operation of the electrode array oxidises or reduces the at least a component of the catalyst substrate to activate the at least a component of the catalyst substrate whereby the at least a component of the catalyst substrate becomes water-soluble.

49. The device of claim 47, wherein the electrode array is configured to detect a catalytic reaction between the catalyst substrate and the catalytic labelling reagent by detecting an amount of a reaction substrate, product, or unreacted catalytic labelling reagent, or any combination thereof.

50. The device of claim 47, wherein the electrode array further has deposited thereon a catalytic moiety.

51. The device of claim 50, wherein the at least a component of the catalyst substrate forms the catalyst substrate when dissolved by the liquid sample on the membrane to enable a catalytic reaction between the catalyst substrate and the catalytic labelling reagent to form a secondary catalyst substrate.

52. The device of claim 51, wherein the secondary catalyst substrate reacts catalytically with the catalytic moiety to form a secondary reaction product.

53. The device of claim 52, wherein the electrode array is configured to detect the secondary reaction product or the secondary catalyst substrate.

54. The device of claim 52, wherein the secondary catalyst substrate comprises one or more oxidation or reduction products capable of being monitored using an electrochemical technique, optionally such as conversion of various substrates to water or one or more oxidised sugars.

55. The device of claim 51, wherein:
the catalytic moiety comprises an enzyme, a nanoparticle, a nanozyme, or a combination thereof; and
the secondary catalyst substrate comprises one or more substrates capable of undergoing a redox reaction, optionally such as hydrogen peroxide, 4-aminophenyl phosphate, or o-aminophenol.

56. The device of claim 44, wherein the device comprises an insulating film disposed between the membrane and the electrode array to chemically separate the membrane from the electrode array.

57. The device of claim 56, wherein the insulating film comprises one or more polymer-based films configured to degrade upon contact with liquid, optionally such as a polysaccharide.

58. The device of claim 56, wherein the support structure comprises a compressible element configured to compress the insulating film upon activation, optionally such as to pierce the insulating film, or to change a physiochemical property.

59. The device of claim 44, wherein the electrode array comprises at least a first pair of corresponding electrodes, the first pair of corresponding electrodes being arranged to overlay the test position on the membrane upon activation, and, optionally, the electrode array comprises a second pair of corresponding electrodes, the second pair of corresponding electrodes being arranged to overlay a background of the membrane.

60. An electronic reader for reading a result from a lateral flow test device, comprising:
a receiving portion configured to receive the lateral flow test device and comprising an activation mechanism configured to activate the lateral flow test device by bringing an electrode array into contact with a membrane; and
a reading port configured to electrically couple with the electrode array to generate an electrical signal in the electrode array and to receive a resulting electrochemical signal through the electrode array.

61. The electronic reader of claim 60, wherein the activation mechanism of the receiving portion brings the electrode array into contact with the membrane by compressing a portion of the lateral flow test device, optionally piercing an insulating film separating the membrane and the electrode array.

62. A method of performing a lateral flow test on a liquid sample using a lateral flow test device, the method comprising:
depositing the liquid sample onto a sample pad of a test strip, wherein the test strip comprises:
a membrane having a first capturing reagent disposed on a first surface at a test position, the first capturing reagent being configured to capture an analyte in the liquid sample;
a sample pad disposed on the first surface at a first end of the membrane spaced apart from the test position, the sample pad being configured to receive the liquid sample;
a conjugate pad disposed on the first surface between the sample pad and the test position, the conjugate pad having deposited thereon a catalytic labelling reagent, the catalytic labelling reagent being configured to chemically bind to the analyte;
an electrode array disposed over the membrane configured to apply an electrical potential across the first surface upon activation, the electrode array having deposited thereon at least a component of a catalyst substrate, the catalyst substrate being capable of chemically reacting with the catalytic labelling reagent upon activation; and
a support structure configured, when the lateral flow test device is in an inactive state, to affix the test strip and the electrode array such that the electrode array is separated from the membrane;
bringing the electrode array into contact with the membrane to activate the lateral flow test device;
operating the electrode array to apply an electrical potential across the first surface through the electrode array to drive an electrochemical reaction in the test strip; and
measuring a resulting electrochemical signal from the test strip through the electrode array to detect a presence of the analyte in the liquid sample.

63. The method of claim 62, wherein measuring a resulting electrochemical signal from the test strip through the electrode array comprises potentiometry, amperometry, voltammetry, one or more impedance-based measurements, one or more capacitive measurements, or a combination thereof.
